(19) United States
(12) Patent Application Publication
(10) Pub. No.: US 2013/0035691 A1
(43) Pub. Date: Feb. 7, 2013

(54) SURGICAL BRUSH

(71) Applicants: Clint Boylan, Minneapolis, MN (US); Steve Wolfe, Eagan, MN (US); Dan McPhillips, Ham Lake, MN (US); Karen Roche, Stillwater, MN (US)

(72) Inventors: Clint Boylan, Minneapolis, MN (US); Steve Wolfe, Eagan, MN (US); Dan McPhillips, Ham Lake, MN (US); Karen Roche, Stillwater, MN (US)

(21) Appl. No.: 13/544,702

(22) Filed: Jul. 9, 2012

Related U.S. Application Data
(60) Provisional application No. 61/506,021, filed on Jul. 8, 2011.

(51) Int. Cl. A61B 17/16

(52) U.S. Cl. 606/84

(57) ABSTRACT

A stainless steel bristled surgical brush for removing nucleus material from the intervertebral space and cartilage from vertebral endplates is provided herein. The surgical brush of the present invention may be dimensioned to be placed through a minimally invasive and/or percutaneous access through an access portal. In certain embodiments, the surgical brush may be straight or bent, may include a cap to contain the tines and may include a depth stop. The brush may be manually operated, powered by a wire driver or powered by other suitable means.

### PRIORITY

This application claims the benefit of U.S. Provisional Application Ser. No. 61/506,021, filed on Jul. 8, 2012, which is hereby incorporated by reference in its entirety herein.

### FIELD

The present invention relates generally to a brush for removing soft tissue. More particularly the present invention relates to a stainless steel bristled brush for removing nucleus material from the intervertebral space and cartilage from vertebral endplates.

### BACKGROUND

During spine surgery it is often necessary to remove soft tissue from the intervertebral space, for example during a discectomy. Such soft tissue may include nucleus material and endplate cartilage. Currently, the soft tissue is generally removed using curettes, rongeurs and disc rakes. The currently available tools are clumsy, making the process of removing the soft tissue arduous. Removing soft tissue is even more difficult in a minimally invasive or percutaneous approach to the spine.

It is desirable to overcome these disadvantages of the currently available tissue removal tools and provide a tool that can predictably remove soft tissue to prepare a surgical site through a minimally invasive and/or percutaneous approach.

### SUMMARY

The surgical brush of the present invention may be dimensioned to be placed through a minimally invasive and/or percutaneous access through an access portal. According to one aspect, the surgical brush of the present invention may be straight. In another embodiment of the present invention the surgical brush may be bent.

In one embodiment of the present invention, the brush may include a cap or sheath adapted to contain the tines. The cap may also provide assist in detecting when the brush has contacted the contra-lateral side of the annulus. In another embodiment of the present invention, the brush may include a depth stop which prevents the brush from moving past the previously drilled depth.

According to one aspect of the present invention, the brush may be manually operated. According to another embodiment of the present invention, the brush may be powered by a wire driver or other powered device.

In one embodiment of the present invention, the brush may be comprised of stainless steel. However other materials, such as plastics and polymers, can be used without departing from the scope of the invention.

In one embodiment, a brush of the present invention may have a bristle diameter in the range of about 0.250-0.600 inches. According to an embodiment of the present invention, the bent brush may be bent at an angle in the range of about 20-30 degrees. In one embodiment, the bent brush may include a bent portion in the range of about 0.250-0.500 inches in length. Other dimensions are contemplated and are within the scope of this invention.

In one aspect of the present invention, the brush bristles may be straight. In another embodiment of the present invention, the brush bristles may be crimped.

According to a bent embodiment of the present invention, the brush sweep increases by about three times over the brush sweep of a straight brush.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a side perspective view of a straight brush according to one embodiment of the present invention.

FIG. 2 depicts a side perspective view of a bent brush according to an embodiment of the present invention.

FIG. 3 depicts a perspective view of a bent brush according to an embodiment of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

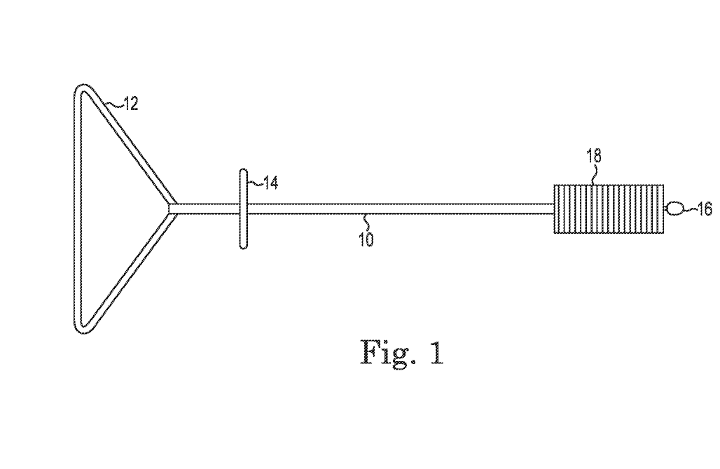

As can be seen in FIG. 1, brush 10 of the present invention may include handle 12. Handle 12 may be a t-handle, a knob or any other desired or suitable handle configuration. Brush 10 may further include depth stop 14. Depth stop 14 may be positioned such that brush 10 is prevented from being inserted into a patient past the original access drill depth.

Brush 10 may further include a sheath, cover or cap 16. Cap 16 holds the tines of brush 10 together and prevents the tines from unraveling. When used in the intervertebral space, cap 16 may also provide a positive feedback indication to the user that the brush has abutted the contra-lateral side of the annulus.

Brush 10 may also include bristles 18 having a bristle diameter in the range of about 0.250-0.600 inches. Bristles 18 may be straight or crimped.

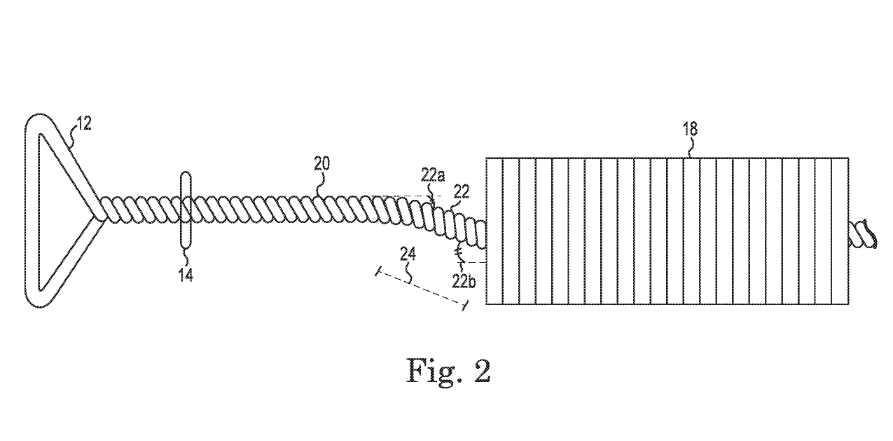

FIG. 2 depicts brush 20 having a bent region 22. Bent region 22 may include angles 22a and 22b. Angles 22a and 22b may be in the range of about 20 to 30 degrees. Bent region 22 may include a bend length 24 in the range of about 0.250-0.500 inches.

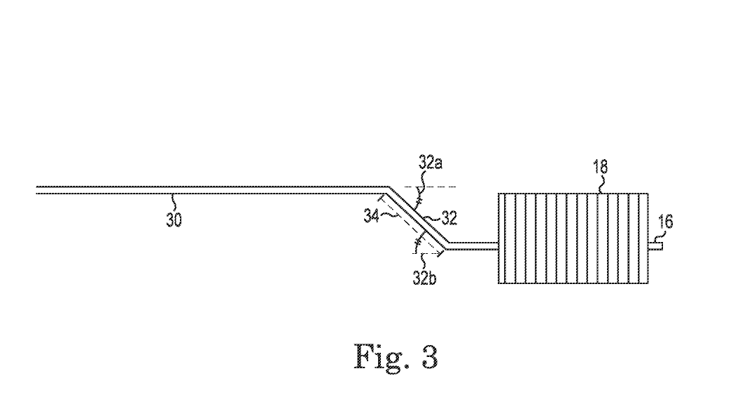

FIG. 3 depicts another bent embodiment of the present invention. Brush 30 may include bent region 32 having angles 32a and 32b. Angles 32a and 32b may be in the range of about 20 to 30 degrees. Bent region 32 may include a bend length 34 in the range of about 0.250-0.500 inches.

Bent regions 22 and 32 may provide the brush of the present invention with about a three times larger sweeping area than in a straight brush embodiment. For example, in a straight embodiment with a bristle diameter of 0.250 inches, the sweeping diameter is the same as the bristle diameter: 0.250 inches. In a bent embodiment with a 0.250 inch bristle diameter, the bent brush provides a sweeping diameter in the range of about 0.850 inches.

Surgical brushes according to the present invention are dimensioned to be placed through a surgical access portal having an internal diameter in the range of about 0.200-0.400 inches. Surgical brushes according to the present invention may be used to remove soft tissue from any surgical site.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A surgical brush configured to remove soft tissue from a surgical site, comprising:
a brush body having a handle operably connected to the distal end of the body;
a brush portion having bristles with a bristle diameter in the range of about 0.25 to 0.6 inches; and
a depth stop.

2. The brush of claim 1, further including a cap configured to maintain the position of the bristles.

3. The brush of claim 1, wherein the bristles are straight.

4. The brush of claim 1, wherein the bristles are crimped.

5. A surgical brush configured to remove soft tissue from a surgical site, comprising:
a brush body having a handle operably connected to the distal end of the body, the brush body including at least one bent region wherein the at least one bent region is at an angle in the range of about 20 to 30 degrees from the longitudinal axis of the brush body; and
a brush portion having bristles.

6. The surgical brush of claim 5, wherein the bent region has a length in the range of about 0.25 to 0.5 inches.

7. The brush of claim 5, further including a cap configured to prevent the bristles from unraveling.

8. The brush of claim 5, wherein the bristles are straight.

9. The brush of claim 5, wherein the bristles are crimped.

10. The brush of claim 5, further including a depth stop.

11. A surgical brush configured to at least one of scrape and score a vertebral endplate, the brush comprising:
a brush body having a handle operably connected to the distal end of the body;
a brush portion having bristles with a bristle diameter in the range of about 0.25 to 0.6 inches; and
a depth stop.

12. The brush of claim 11, further including a cap configured to maintain the position of the bristles.

13. The brush of claim 11, wherein the bristles are straight.

14. The brush of claim 11, wherein the bristles are crimped.

15. A surgical brush configured to at least one of scrape and score a vertebral body, the brush comprising:
a brush body having a handle operably connected to the distal end of the body, the brush body including at least one bent region wherein the at least one bent region is at an angle in the range of about 20 to 30 degrees from the longitudinal axis of the brush body; and
a brush portion having bristles.

16. The surgical brush of claim 15, wherein the bent region has a length in the range of about 0.25 to 0.5 inches.

17. The brush of claim 15, further including a cap configured to prevent the bristles from unraveling.

18. The brush of claim 15, wherein the bristles are straight.

19. The brush of claim 15, wherein the bristles are crimped.

20. The brush of claim 15, further including a depth stop.
